(19) United States
(12) Patent Application Publication
(10) Pub. No.: US 2005/0214345 A1
(43) Pub. Date: Sep. 29, 2005

(54) Artificial biocompatible material as a support for cells in a retinal implant

(76) Inventors: Theodore Leng, Fallerton, CA (US); Dimitri Yellachich, Berkshire (GB); Philip Huie JR., Cupertino, CA (US); Jaan Noolandi, Palo Alto, CA (US); Harvey A. Fishman, Menlo Park, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/061,171

(22) Filed: Feb. 18, 2005

Related U.S. Application Data
(60) Provisional application No. 60/546,392, filed on Feb. 20, 2004.

(51) Int. Cl. A61F 2/14; A61K 31/716

(52) U.S. Cl. 424/427; 623/4.1

(57) ABSTRACT

A retinal implant is provided that uses an artificial biocompatible material as a support material on which retinal pigment epithelial cells, iris pigment epithelial cells, and/or stem cells can be deposited either in situ or in vivo. The support material has a surface topology that is rough to promote cell adhesion, has surface pits to allow pigment cells to grow into, and has pores to allow for proper diffusion of materials. The support material serves as a substrate for cell growth and as a patch for damaged basement membrane (Bruch's membrane). This cell-coated membrane or pigment cell-enriched membrane is surgically positioned in the sub-retinal space to rescue or restore photoreceptor cell function that may be damaged or threatened by degenerative diseases of the eye, such as age-related macular degeneration.

### CROSS-REFERENCE TO RELATED APPLICATIONS

This application is cross-referenced to and claims priority and the benefit from U.S. Provisional Application 60/546,392 filed Feb. 20, 2004, which is hereby incorporated by reference.

### FIELD OF THE INVENTION

The present invention relates generally to the field of treatment of eye disorders. More particularly, the present invention relates to implants for retinal disorders such as age-related macular degeneration.

### BACKGROUND

Diseases of the retina, such as age-related macular degeneration (AMD) are the leading cause of severe visual impairment or blindness in elderly patients in the industrialized world. Although the exact pathogenesis of AMD is unknown, one important factor involves the death of retinal pigment epithelial (RPE) cells at the posterior of the eye, underneath the retina in the sub-retinal space. The RPE basement membrane (Bruch's membrane) is also damaged in AMD by mechanisms that include oxidative damage, and allows for new blood vessel growth. Death of the photoreceptor cells, and eventual blindness, follows death of the RPE cells.

One theory suggests that replacement of dying RPE cells in the sub-retinal space may rescue or restore function(s) to the photoreceptor cells. First attempts at RPE cell transplantation involved injecting a suspension of RPE cells into a patient's sub-retinal space. This approach was supplanted by transplant of intact sheets of RPE cells. Each of these techniques was plagued with problems arising from disorientation of the transplanted cells, inability of these cells to spontaneously form an organized monolayer, ineffectiveness to perform phenotypic functions of native RPE cells, and from continuing destruction of the Bruch's membrane in the AMD process. It has been suggested that transplanted RPE cells also perform poorly in the pathological sub-retinal space because such cells attach poorly to a damaged Bruch's membrane of eyes affected by AMD. Transplantation of cell suspensions, or even of patches or confluent sheets of such cells, may therefore be ineffective for AMD treatment.

Another possible approach is to grow RPE, iris pigment epithelium (IPE), and/or stem cells on a suitable support material and to transplant both the cells and the support material into the sub-retinal space. Several groups have studied different materials, such as anterior lens capsule and Descemet's membrane, for transplantation of RPE cells and IPE cells into the sub-retinal space. These attempts have been unsuccessful because of the handling properties of the support materials used by these experimenters. Although cells have been grown on lens capsule, it is difficult to implant lens capsule into the sub-retinal space, due to its tendency to curl on itself, especially in an aqueous environment like the eye. It is an even greater challenge to maintain lens capsule material flat when the material is implanted into the sub-retinal space. Furthermore, the growth properties and related characteristics of pigment epithelial cells are greatly influenced by the surface properties of a support material on which the cells might grow.

Accordingly, there is need in the art for new types of implants and methods for the treatment of retinal diseases such as AMD. It would be desirable to have an implantable support material that (i) is biocompatible, (ii) will serve as a growing surface for selected biological substances, (iii) has a controllable range of porosity, and/or (iv) will not spontaneously roll up, form creases or tear during surgery and once implanted into the subretinal space.

### SUMMARY OF THE INVENTION

The present invention provides a retinal implant with an artificial biocompatible support material for RPE, IPE and/or stem cells that can take on the function of RPE. The surface topology of the support material of the retinal implant is characterized by being rough, which promotes cell adhesion, having surface pits to allow pigment cells to grow into, and having pores to allow for proper diffusion of nutrients, waste, oxygen, and carbon dioxide. In general, the support material could be any type of artificial biocompatible material having such characteristics. An example of such a material is for instance an unmodified dialysis membrane of about a 100 kD molecular weight cutoff (MWCO). The characteristics could also, for instance, be etched-into the support material for instance by using an 193 nm excimer laser. Examples of suitable materials are, for instance, but not limited to, cellulose, cellulose acetate or any derivative thereof, cellulose acetate ester dialysis membrane, silicon, polyester or any synthetic polymer provided that it has the physical characteristics as described herein. An additional characteristic for the support material is that it is relatively rigid and remains flat in an aqueous environment, but can be made flexible enough to conform to the inner retina or any other space that hosts the implant. The material is preferably non-soluble when present in a subretinal space and would therewith remain as a support of the cells.

In one aspect the support material is about 1 micron to about 150 microns thick. In another embodiment the thickness is up to about 100 microns. The material has pores sized to allow diffusion of nutrients, waste, oxygen and carbon dioxide, and has surface pits about 0.05 microns to about 1 micron in diameter to allow cells to grow into it. These surface pits could meander up to about 5 microns from the surface into the support material, typically on the order of 2-3 microns, however they could also be completely through the thickness of the support material. For a retinal implant in the foveal region the implant could be a single sheet of material with an area of about, but not limited to, 9 mm². However, the present invention is not limited to such an area and could be small or larger depending on the area/space that requires restoration or repair.

The cells are grown on the support material either in situ or in vivo and will typically and preferably arrange themselves on the surface in a monolayer. In another aspect, the cells could be arranged in a pattern on the surface of the support material. Such a pattern could be established by means of microcontact printing, means of soaking or means of coating of inhibitory molecules, adhesive molecules or inhibitory molecules and adhesive molecules. Examples of patterns are triangles, quadrilaterals, pentagons, hexagons, n-sided polygons with n at least equal to 7, circles and ovals.

In one example the method of implanting the retinal implant, i.e. the pigment cell-enriched support material, could be surgically transplanted into a selected region in the sub-retinal space by insertion through an aperture created in the selected region using a sharp instrument (e.g. a knife) after the selected region of the retina has been elevated through an injection of a physiologically appropriate solution such as a balanced salt solution. After the insertion the selection region could be flattened out through the use of a heavy fluid like perfluro-carbon and/or the use of air-fluid exchange.

The advantages of the present invention are that the support material of the present invention will allow for improved precision during surgical handing and will remain relatively flat against the choroid when the combination of support material and RPE, IPE, and/or stem cells are transplanted into the sub-retinal space. The support material of the present invention will serve simultaneously as a cell growth substrate and as a “patch” for damaged Bruch's membrane to prevent the growth of unwanted blood vessels into the retina. Another advantage is that the growth and support of cells could be accomplished without a surface modifier such as collagen or the like.

### BRIEF DESCRIPTION OF THE FIGURES

The present invention together with its objectives and advantages will be understood by reading the previous summary and following description in conjunction with the drawings, in which:

FIG. 1 shows a light micrograph of a growth of chick RPE/retina co-culture on 100K MWCO cellulose acetate ester dialysis membrane, in cross section, after six days in culture. Both retina and RPE are viable. RPE retain pigment granules.

FIG. 2 shows a light micrograph of a full thickness retina/RPE, in cross section, grown on top of 100K MWCO cellulose ester dialysis membrane. A sheet of chick RPE was harvested on day 0 and allowed to adhere to the dialysis membrane. On day 3, freshly prepared chick retina was placed on top of the established and adherent RPE layer. The RPE/retina was cultured for an additional 3 days. This complex was fixed on day 6 with glutaraldehyde, embedded in LX-112 resin, sectioned for light microscopy, and stained with toluidine blue. The RPE cells maintained their pigment granules and the RPE appeared to interact with the overlaying retina. The RPE grew as a single layer in some areas and had the appearance of an epithelial phenotype.

FIG. 3 shows a light micrograph looking through a RPE sheet grown on dialysis membrane with overlying whole retina (in vitro). The RPE was transferred as a sheet and incubated on 100K MWCO dialysis membrane (CE). Note the close packing of the RPE cells and the retention of pigment granules. This is a 6-day co-culture. LM section of this material is shown in FIGS. 1-2.

FIG. 4 shows a transmission electron microscopy analysis (TEM) of a cross section through a dialysis membrane with RPE cells adhered to its surface. It is noted that the surface is rough and RPE cytoplasm is in the pit of the dialysis membrane.

FIGS. 5-10 shows atomic force microscope analyses of the front side (FIGS. 5-7, 9; “good”) versus analyses of the back side (FIGS. 8, 10; “bad”) of a 100K MWCO dialysis membrane. The front ‘good’ side appears to have surface pits on the order of 0.05 micron to 1 micron in diameter. Larger pits (1.0-2.0 micron) are also present in the ‘bad’ side. Cells will eventually grow on the bad side however the good side promotes cell growth, which is faster than on the bad side. What distinguishes the good side from the bad side are the presence of smaller pits that meander from the surface to about 4-5 microns below the surface of the ‘good’ side and have dimensions of about 0.05 to 1.0 microns. Since in this example the support material is obtained from a dialysis membrane a definition is required to define the front side and back side: i.e. the front side refers to the outside of the tube of the dialysis membrane hence the back side refers to the inside of the tube of the dialysis membrane.

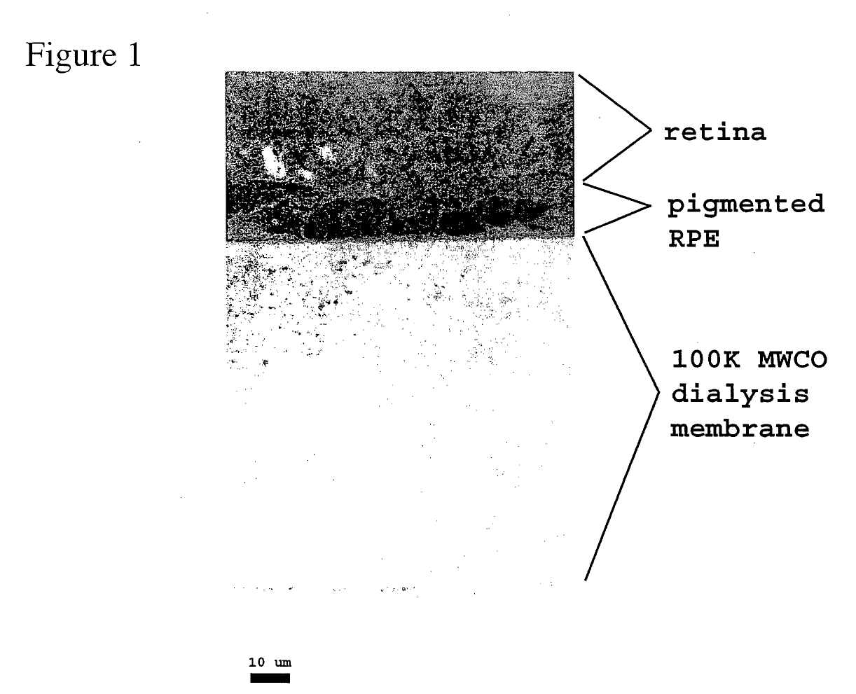

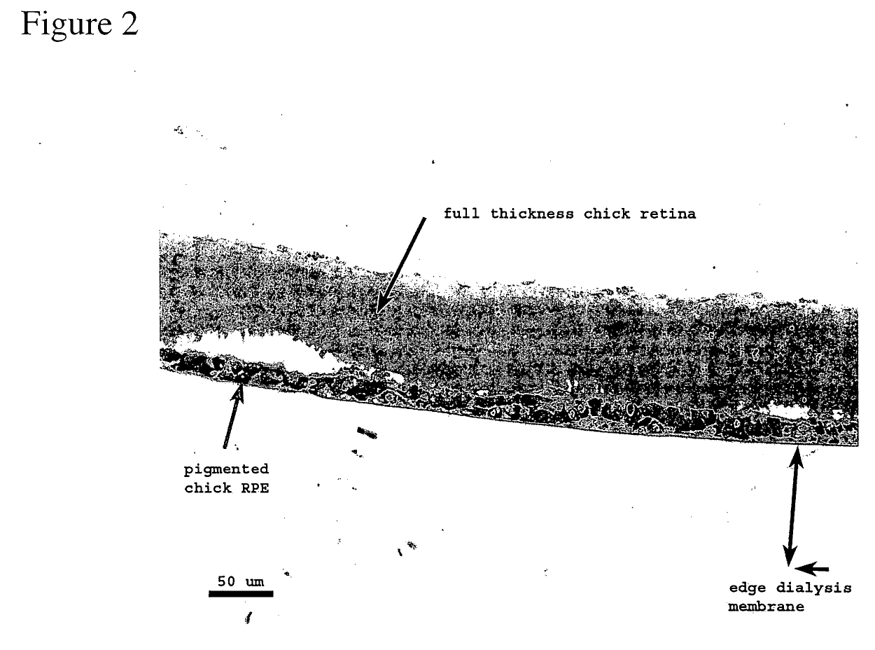

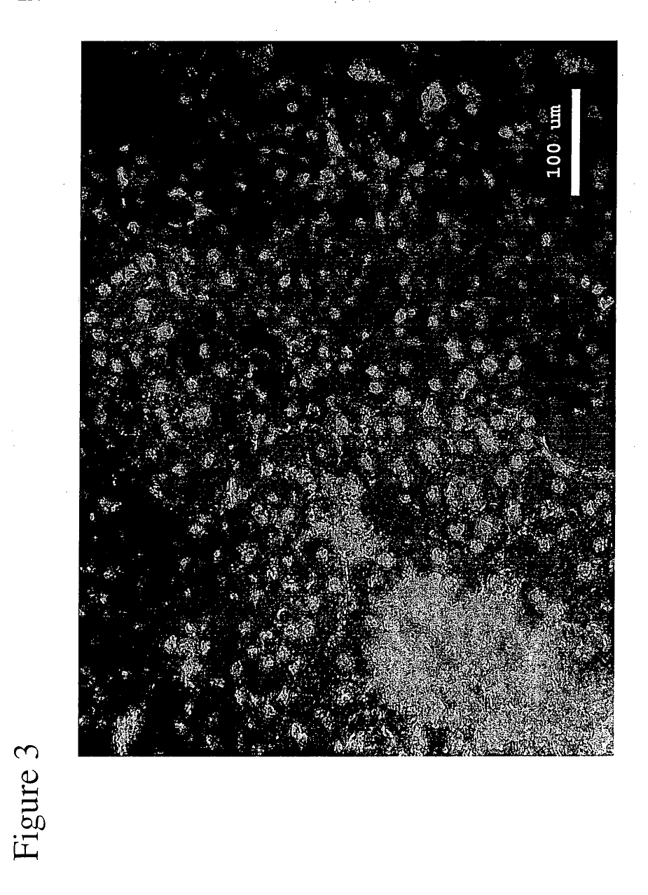

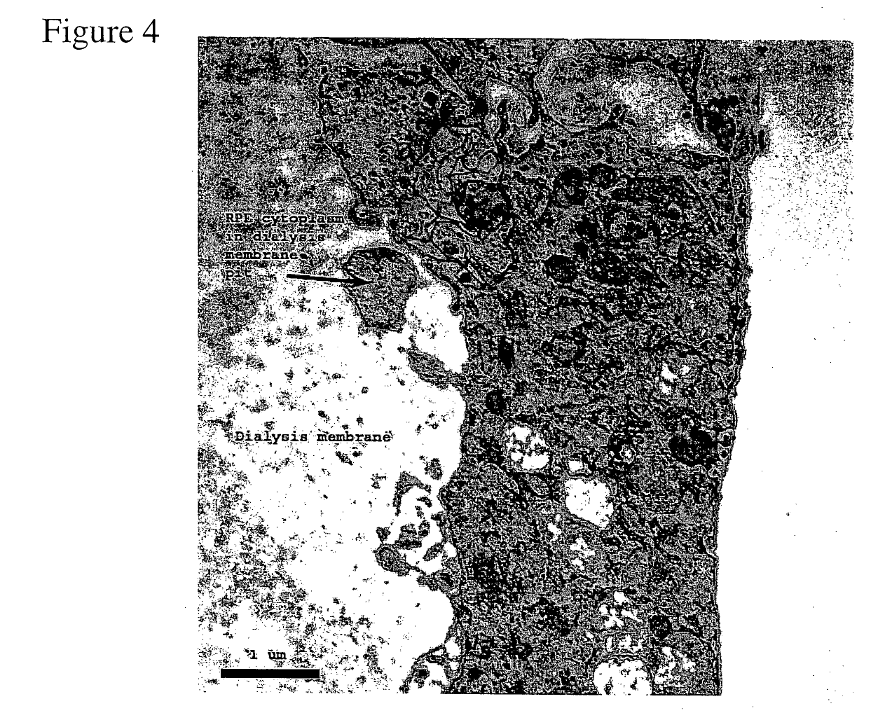

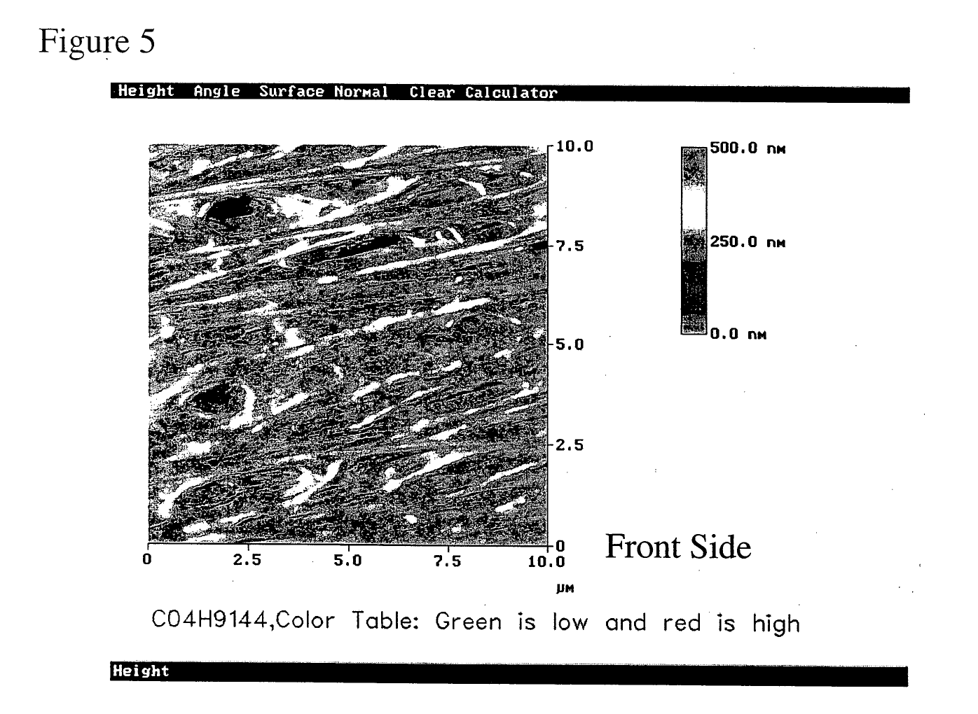

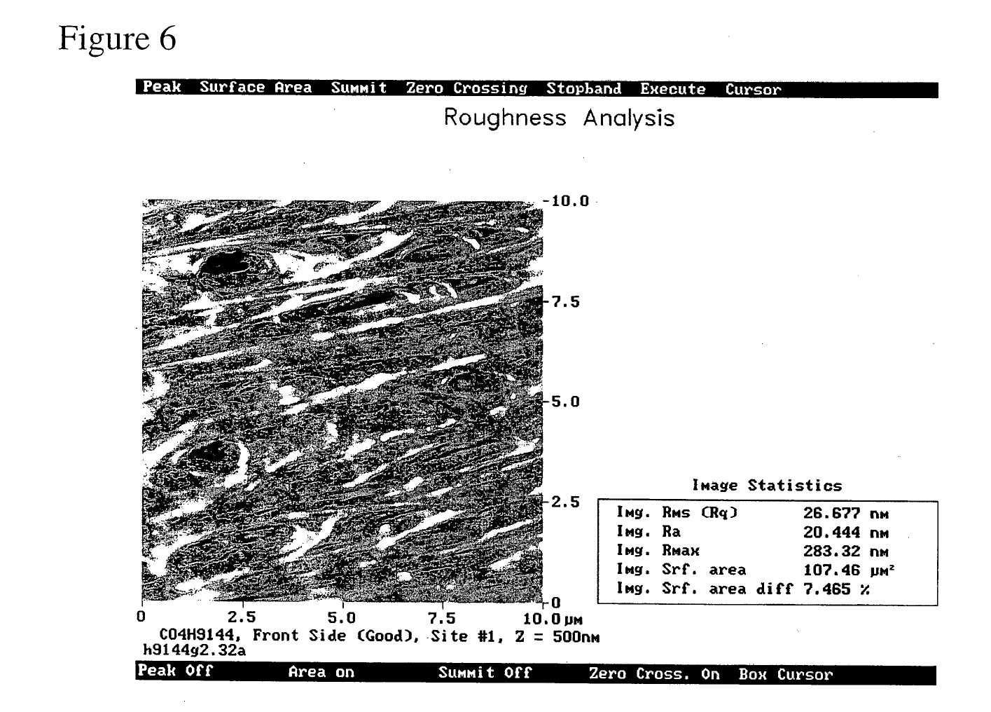

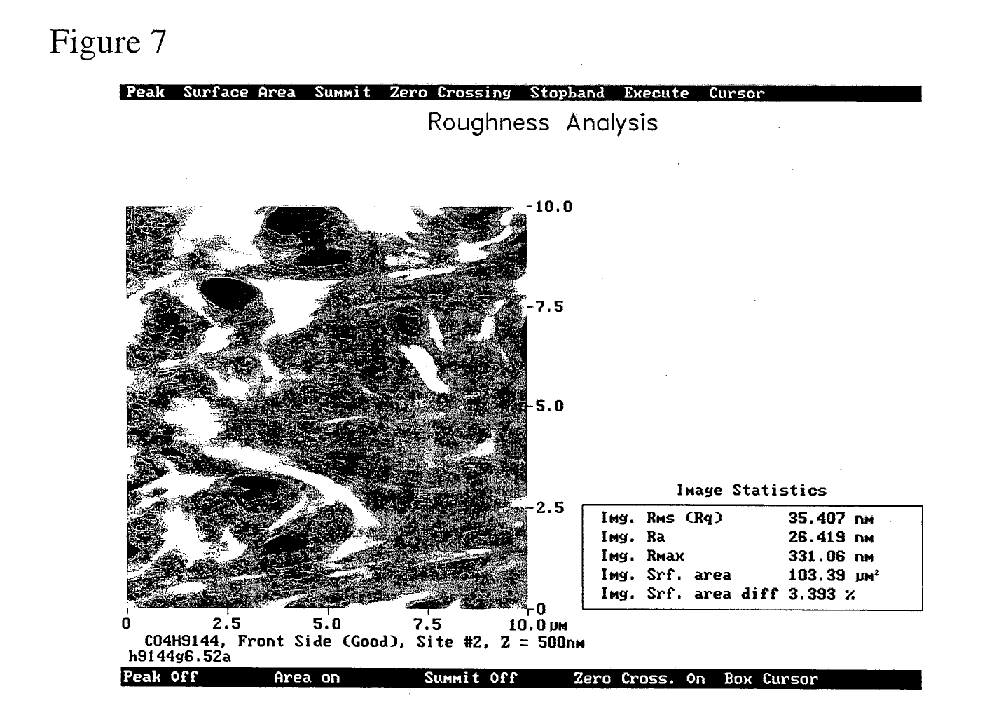

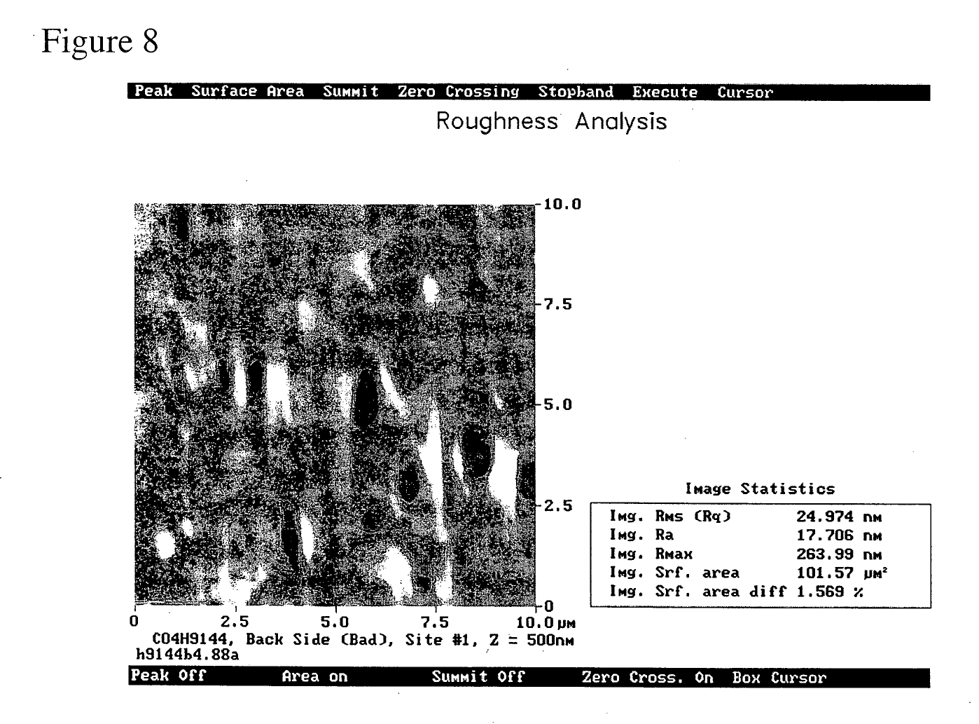

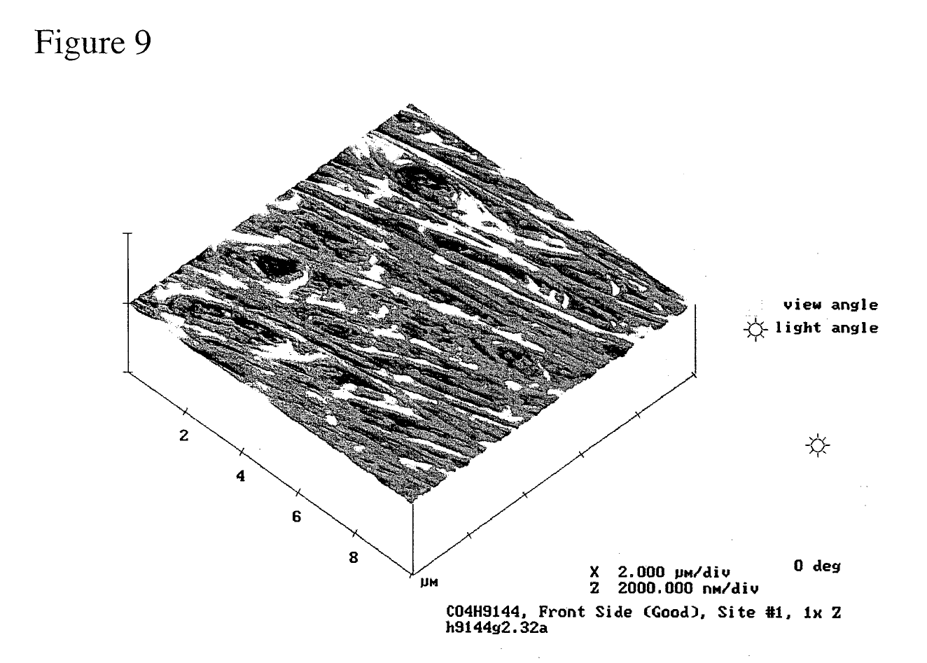

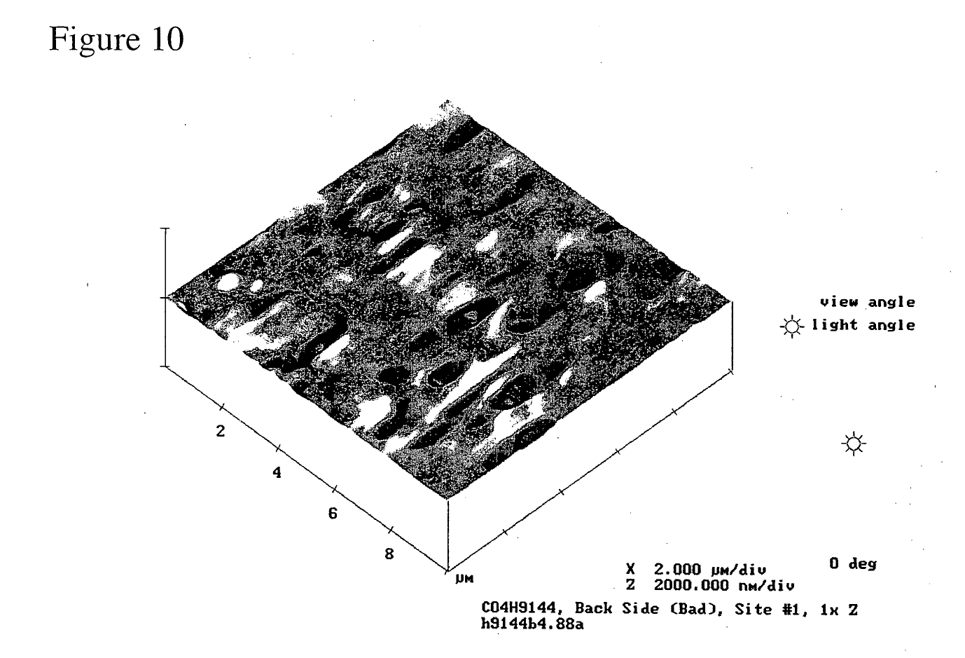

### DETAILED DESCRIPTION OF THE INVENTION

The present invention provides an implant to rescue or restore diseased photoreceptor cells, using transplantation of RPE, IPE, and/or stem cells adhered on an artificial biocompatible support material to the sub-retinal space of the eye. The artificial biocompatible support material serves both as a transplant/support material and as a basement membrane (Bruch's membrane) patch. Even though the following detailed description refers to a dialysis membrane as the artificial biocompatible support material, the present invention is not limited to the dialysis membrane as described supra in the summary and description of the figures. Furthermore, the cells or stem cells used for this invention could be of human or animal origin as well as possibly Xenobiotic organ, tissue and cell transplants.

Cell Culture

Human RPE cells for transplantation were maintained in D-MEM/F-12 solution, supplemented with 10 percent fetal bovine serum at T=37 degrees Celsius with 6.5 percent carbon dioxide. The cells were removed from 100 mm tissue culture dishes with 0.05 percent trypsin-EDTA and were cultured at a 1:10 ratio. A concentration of 10⁶ cells/L was cultured onto sterilized dialysis membrane.

Animal IPE cells were harvested and isolated from New Zealand Red or hybrid rabbits, using an enzyme-assisted microdissection procedure modified from that which is described by Hu and McCormick, in Archives of Ophthalmology, Vol 115 (1997) pp. 89-94. The primary culture was maintained in F-12 nutrient mixture (HAM) with L-glutamine supplemented with 20 percent fetal bovine serum and 50 μg/mL gentamycin. The maintenance media was exchanged every three days.

Surgical Technique

A dialysis membrane was implanted into the sub-retinal space of New Zealand Red and hybrid rabbits, each weighing 2.5-3.5 kg. The rabbits were anesthetized with ketamine (40 mg/kg) and Xylazine (5 mg/kg), administered through intramuscular injection. One dose of Tropicamide (0.5 percent) eye drops and Phenylephrine (2.5 percent) eye drops were instilled into the conjunctival sac of the left eye. A standard three-port pars plana vitrectomy was performed, and a retinal bleb was inflated in the macular area by injection of approximately 0.5 mL of balanced salt solution (BSS) through a 41-gauge needle. A 1 mm retinotomy was created using a MVR blade, and the dialysis membrane was inserted into the sub-retinal space through the aperture. Perfluro-carbon heavy fluid was used to flatten the retina followed by a perfluro-carbon silicone oil exchange. The care of animals conformed to the ARVO Statement of the Use of Animals in Ophthalmic and Vision Research, and the Administrative Panel on Laboratory Animal Care at Stanford University approved the protocol (No. 6597) employed.

Histology

Rabbit eyes were enucleated one, two, and four weeks after implantation. The eyes were fixed in 1.25 percent gluteraldehyde/l percent paraformaldehyde in cacodylate buffer (pH=7.4). After fixation, the eyes were cut open, fixed, post-fixed in osmium tetroxide, dehydrated with a graded series of EtOH, and embedded in epoxy resin. Sections of 1 micron thickness were stained with toluidine blue for improved contrast.

Dialysis Membrane Patterning

A pattern (or array) for RPE, IPE, and/or stem cell growth can be defined. The pattern could be established by means of microcontact printing, means of soaking or means of coating of inhibitory molecules, adhesive molecules or inhibitory molecules and adhesive molecules. Such techniques are common and known in the art, however the present invention is not limited to these techniques since other or newly developed patterning techniques could also be used. Examples of different possible patterns are triangles, quadrilaterals, pentagons, hexagons, n-sided polygons with n at least equal to 7, circles and ovals.

In a specific example, a cell growth inhibitor, such as polyvinyl acid (PVA), can be stamped onto the surface of the dialysis membrane using the technique of microcontact printing (soft lithography) to influence the surface growth properties of the cells. Thus, the cultured RPE, IPE, and/or stem cells may more closely resemble the morphology of RPE in vivo. Poly(dimethylsiloxane) (PDMS) stamps can be prepared as described by Whitesides G M, Ostuni E, Takayama S, Jiang X, and Ingber D E, Annu Rev Biomed Eng., Vol 3, (2001), pp. 335-373. Briefly, a chrome mask with the desired microscale pattern was fabricated at the Stanford Nanofabrication Facility. The mask was used to pattern a 7 μm layer of SPR-220 photoresist (Shipley, Marlborough, Mass.) on a silicon wafer. PDMS (Sylgard 184; Dow Corning Corporation, Midland, Mich.) in a 10:1 mixture of elastomer to curing agent was then poured onto the patterned silicon wafer and cured at 90° C. After one hour, the PDMS stamp was removed from the patterned silicon wafer to reveal the desired microscale pattern. PDMS stamps (1 cm²) were placed in a plasma cleaner/sterilizer (PDC-32G; Harrick Scientific Corporation, Ossining, N.Y.) for 1 minute at 100 W to obtain a hydrophilic surface. Stamps were sterilized by immersion in 70% ethanol for 1 minute and dried with nitrogen. To stamp a surface, the PDMS stamp can be placed carefully onto a thin layer of either 5% mucilage (Elmer's Products Incorporated, Columbus, Ohio) in distilled water or 2% polyvinyl alcohol (PVA) in distilled water. In both cases, the solution is supplemented with 0.1 mg/mL of fluorescein for imaging. Immediately after contact, the PDMS stamp is removed from the thin layer of solution and placed in contact with the surface to “wet transfer” the solution. A 40-g weight is placed on top of the stamp for 30 minutes after which the microprinted surface is ready for cell culture.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations and other variations are considered to be within the scope and spirit of the present invention as defined by the following claims and their legal equivalents.

1. An retinal implant, comprising: a single layer of an artificial biocompatible material having pores to allow diffusion through said material and surface pits to allow anchoring of the cell processes of said cells,
wherein said material is about 1 micron to about 150 micron thick, and
wherein said surface pits have a diameter of about 0.05 micron to about 1 micron and meander up to about 5.0 micron from said surface into said material.

2. The retinal implant as set forth in claim 1, wherein said material is up to about 100 micron thick.

3. The retinal implant as set forth in claim 1, wherein said material is a cellulose acetate or any derivative thereof, a cellulose acetate ester dialysis membrane, a silicon, a polyester, or a synthetic polymer.

4. The retinal implant as set forth in claim 1, wherein said material is a cellulose ester dialysis membrane of about 100 kD MWCO.

5. The retinal implant as set forth in claim 1, wherein said retinal implant is in a subretinal space.

6. The retinal implant as set forth in claim 1, wherein said material is flexible to conform to the surface of a subretinal space.

7. The retinal implant as set forth in claim 1, wherein said pores are sized to allow diffusion of nutrients, waste, oxygen and carbon dioxide.

8. The retinal implant as set forth in claim 1, wherein the surface of said material is rough.

9. The retinal implant as set forth in claim 1, wherein said cells are received on the surface of said material in situ or in vivo.

10. The retinal implant as set forth in claim 1, wherein said cells are selected from the group consisting of RPE cells, IPE cells and stem cells that can take on the functional role of RPE cells.

11. The retinal implant as set forth in claim 1, wherein said cells are arranged on the surface of said material in a monolayer.

12. The retinal implant as set forth in claim 1, wherein said cells are arranged on the surface of said material in a pattern.

13. The retinal implant as set forth in claim 12, wherein said pattern is established by means of microcontact printing, means of soaking or means of coating of inhibitory molecules, adhesive molecules or inhibitory molecules and adhesive molecules.

14. The retinal implant as set forth in claim 12, wherein said pattern is selected from the group of patterns consisting of triangles, quadrilaterals, pentagons, hexagons, n-sided polygons with n at least equal to 7, circles and ovals.

15. A method of using a single layer of an artificial biocompatible material as a retinal implant, said material having pores to allow diffusion through said material and surface pits to allow anchoring of the cell processes of said cells,
wherein said material is about 1 micron to about 150 micron thick,
wherein said surface pits have a diameter of about 0.05 micron to about 1 micron and meander up to about 5.0 micron from the surface into said material, and
wherein said cells are selected from the group consisting of RPE cells, IPE cells and stem cells that can take on the functional role of RPE cells.

16. The method as set forth in claim 15, wherein said material is up to about 100 micron thick.

17. The method as set forth in claim 15, wherein said material is a cellulose acetate or any derivative thereof, a cellulose acetate ester dialysis membrane, a silicon, a polyester, or a synthetic polymer.

18. The method as set forth in claim 15, wherein said material is a cellulose ester dialysis membrane of about 100 kD MWCO.

19. A method of surgically inserting a retinal implant in a sub-retinal space, comprising:
(a) surgically inserting a pigment-enriched support material of claim 1, into a selected region in said sub-retinal space through an aperture created in said selected region using a sharp instrument after said selected region of the retina has been elevated through an injection of a physiologically appropriate solution; and
(b) flattening out said selected region of retina through the use of perfluro-carbon heavy fluid or the use of air-fluid exchange.
